(19) United States
(12) Patent Application Publication
(10) Pub. No.: US 2010/0217082 A1
(43) Pub. Date: Aug. 26, 2010

(54) COVERED-TYPE ENDOSCOPE, COVER-ADAPTED ENDOSCOPE AND ENDOSCOPE-COVER

(75) Inventors: Yoshiaki ITO, Fuchu-shi (JP); Hideya Kitagawa, Hachioji-shi (JP)

Correspondence Address: Frishauf, Holtz Goodman & Chick, P.C., 16th Floor, 220 Fifth Avenue, New York, NY, 10001-7708, US

(73) Assignee: Olympus Corporation, Tokyo (JP)

(21) Appl. No.: 12/774,967

(22) Filed: May 6, 2010

Related U.S. Application Data
(63) Continuation-in-part of application No. PCT/JP2008/069258, filed on Oct. 23, 2008.

(30) Foreign Application Priority Data
Nov. 15, 2007 (JP) 2007-296995

(51) Int. Cl. A61B 1/00

(52) U.S. Cl. 600/121

(57) ABSTRACT

A covered-type endoscope includes an endoscope-cover and a cover-adapted endoscope to be inserted into the endoscope-cover. The endoscope-cover includes a tube and a cover-bending portion with a plurality of longitudinally joined cover-joint pieces. The cover-adapted endoscope includes a bending portion with a plurality of longitudinally joined joint pieces. The bending portion includes a guide portion which guides the tube in the longitudinal direction of the bending portion and cooperates with the cover-bending portion of the endoscope-cover to provide a tube placement chamber between the guide portion and the cover-bending portion of the endoscope-cover so that a conduit for the tube is maintained. The tube is flexible to bend in accordance with the bending of the cover-bending portion and the bending portion.

### CROSS-REFERENCE TO RELATED APPLICATIONS

This is a Continuation Application of PCT Application No. PCT/JP2008/069258, filed Oct. 23, 2008, which was published under PCT Article 21(2) in Japanese.

This application is based upon and claims the benefit of priority from prior Japanese Patent Application No. 2007-296995, filed Nov. 15, 2007, the entire contents of which are incorporated herein by reference.

### BACKGROUND OF THE INVENTION

1. Field of the Invention

This invention relates to a covered-type endoscope, a cover-adapted endoscope and an endoscope-cover.

2. Description of the Related Art

For example, an endoscope for surgical use communicates with the outside from an opening at the distal end of an insertion portion that opens in a body cavity of a patient, via a tube inserted through the insertion portion. Therefore, the inside of a conduit for the tube including the opening has to be completely cleaned and disinfected. The work for a user to completely clean and disinfect the inside of the conduit is troublesome and takes much time, and thus decreases the efficiency of the use of the endoscope.

As one solution, there has been proposed a so-called covered-type endoscope wherein a bending portion of the endoscope is covered with an endoscope-cover and inserted into a body cavity, and the endoscope-cover is disposed of every case to simplify the cleaning, disinfection and sterilization of the endoscope after use. In the covered-type endoscope, a distal opening which is open in the body cavity of a patient and tubes are provided on the side of the endoscope-cover, while observation means and illumination means which have no openings and tubes and which are easy to clean, disinfect and sterilize are provided on the side of the cover-adapted endoscope.

One problem peculiar to the covered-type endoscope is letting the cover-adapted endoscope into and out of the endoscope-cover. For example, a disclosure of Jpn. Pat. Appln. KOKAI Publication No. 6-319679 makes it easier to let a cover-adapted endoscope into and out of an endoscope-cover and prevents the abrasion and deterioration of a resin cover that forms a bending portion of the cover-adapted endoscope.

There is another problem. The cover-adapted endoscope that is covered with the endoscope-cover is inserted into the body cavity of a patient, and then an examination and surgery are performed. Therefore, when the insertion portion of the covered-type endoscope is twisted about its axis to bring the observation means of the cover-adapted endoscope into a desired direction, a gripped portion of the endoscope-cover and the insertion portion of the cover-adapted endoscope slip on each other. As a result, the endoscope-cover alone is twisted, and no twisting force is transmitted to the insertion portion of the cover-adapted endoscope. To solve this problem, for example, Jpn. Pat. Appln. KOKAI Publication No. 8-47476 has disclosed a covered-type endoscope in which joint pieces are provided on a cover side to improve twist operability.

### BRIEF SUMMARY OF THE INVENTION

One aspect of a covered-type endoscope according to the present invention includes an endoscope-cover and a cover-adapted endoscope to be inserted into the endoscope-cover. The endoscope-cover includes a tube and a cover-bending portion with a plurality of longitudinally joined cover-joint pieces. The cover-adapted endoscope includes a bending portion with a plurality of longitudinally joined joint pieces. The bending portion includes a guide portion which guides the tube in the longitudinal direction of the bending portion and cooperates with the cover-bending portion of the endoscope-cover to provide a tube placement chamber between the guide portion and the cover-bending portion of the endoscope-cover so that a conduit for the tube is maintained. The tube is flexible to bend in accordance with the bending of the cover-bending portion and the bending portion.

### BRIEF DESCRIPTION OF THE SEVERAL VIEWS OF THE DRAWING

FIG. 1A is a schematic perspective view showing the separated state of a cover-adapted endoscope and an endoscope-cover of a covered-type endoscope according to a first embodiment;

FIG. 1B is a schematic perspective view showing how the endoscope-cover is inserted in the cover-adapted endoscope to configure the covered-type endoscope;

FIG. 2 is a schematic perspective view showing an observation optical system and a bending portion of the cover-adapted endoscope of the covered-type endoscope according to the first embodiment;

FIG. 3 is a schematic front view showing a joint piece of the bending portion of the cover-adapted endoscope of the covered-type endoscope according to the first embodiment;

FIG. 4A is a schematic perspective view showing the observation optical system of the cover-adapted endoscope of the covered-type endoscope according to the first embodiment;

FIG. 4B is a schematic longitudinal sectional view of a distal imaging unit of the observation optical system shown in FIG. 4A;

FIG. 5A is a schematic perspective view of the endoscope-cover of the covered-type endoscope according to the first embodiment;

FIG. 5B is a schematic front view showing an operation portion coupling portion of the endoscope-cover viewed from the side of an operation portion of the cover-adapted endoscope;

FIG. 5C is a schematic perspective view of the operation portion coupling portion of the endoscope-cover;

FIG. 6 is a schematic longitudinal sectional view showing a connection between a cover connecting portion and a cover flexible pipe in the endoscope-cover of the covered-type endoscope according to the first embodiment;

FIG. 7 is a schematic perspective view showing the cover-adapted endoscope of the covered-type endoscope that is halfway inserted in or removed from the endoscope-cover, according to the first embodiment;

FIG. 8 is a schematic perspective view showing, in a partially transmitted form, the cover-adapted endoscope of the covered-type endoscope that is attached to the endoscope-cover, according to the first embodiment;

FIG. 9 is a schematic partial cross-sectional view showing the bending portion of the cover-adapted endoscope that is disposed in a bending portion of the endoscope-cover when the cover-adapted endoscope of the covered-type endoscope is attached to the endoscope-cover, according to the first embodiment;

FIG. 10A is a schematic perspective view showing an observation optical system and a bending portion of a cover-adapted endoscope of a covered-type endoscope according to a second embodiment;

FIG. 10B is a schematic front view of a joint piece shown in FIG. 10A;

FIG. 10C is a schematic front view of a guide portion shown in FIG. 10A;

FIG. 11A is a schematic perspective view showing a cover distal portion, a cover bending portion and a cover connecting portion of an endoscope-cover when the endoscope-cover is inserted through the cover-adapted endoscope of the covered-type endoscope, according to the second embodiment;

FIG. 11B is a schematic partial cross-sectional view showing the guide portion of the bending portion of the cover-adapted endoscope that is disposed within the cover connecting portion;

FIG. 12A is a schematic perspective view showing how an endoscope-cover is inserted in a cover-adapted endoscope of a covered-type endoscope and how torque transmission protrusions formed on the inner surface of a rearmost cover joint piece of a cover bending portion of the endoscope-cover by cutting and bending are engaged with suction tube grooves of a rearmost cover joint piece of a bending portion of a cover-adapted endoscope, according to a third embodiment;

FIG. 12B is a schematic front view showing the rearmost cover joint piece of the cover bending portion of the endoscope-cover; and

FIG. 12C is a schematic front view showing how the torque transmission protrusions formed by cutting and bending shown in FIG. 12A are produced by, for example, a forging process and disposed.

### DETAILED DESCRIPTION OF THE INVENTION

A best mode for carrying out the invention (hereinafter referred to as an embodiment) will be described hereinafter with reference to the drawings.

### First Embodiment

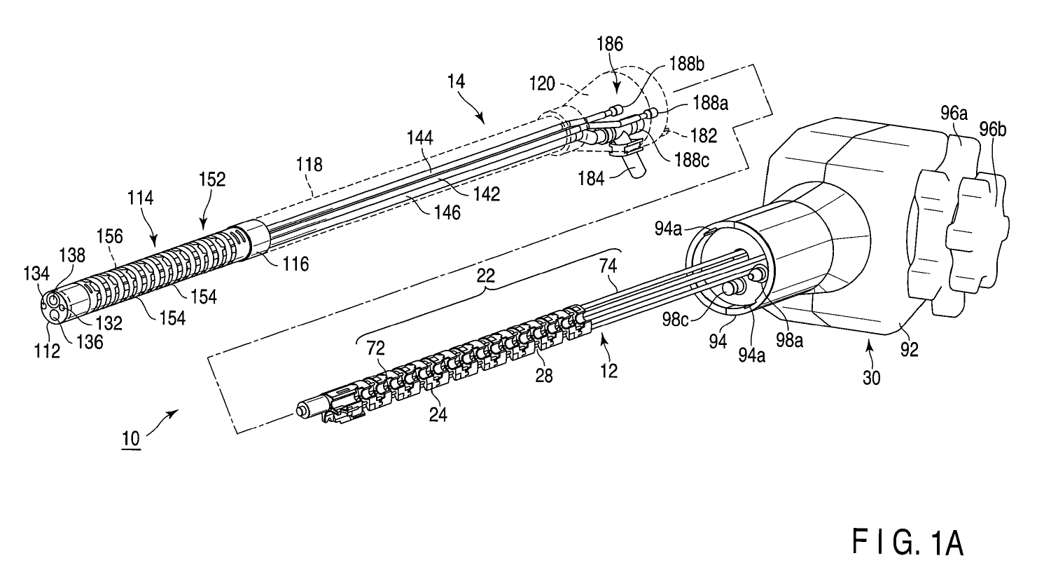

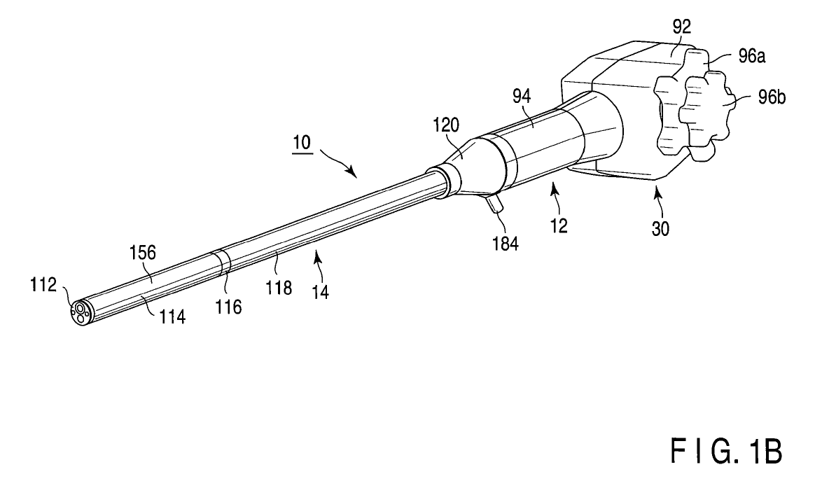

A first embodiment is described with reference to FIGS. 1A to 9.

As shown in FIGS. 1A and 1B, a covered-type endoscope 10 includes a cover-adapted endoscope 12 and an endoscope-cover 14 in an attachable/removable manner.

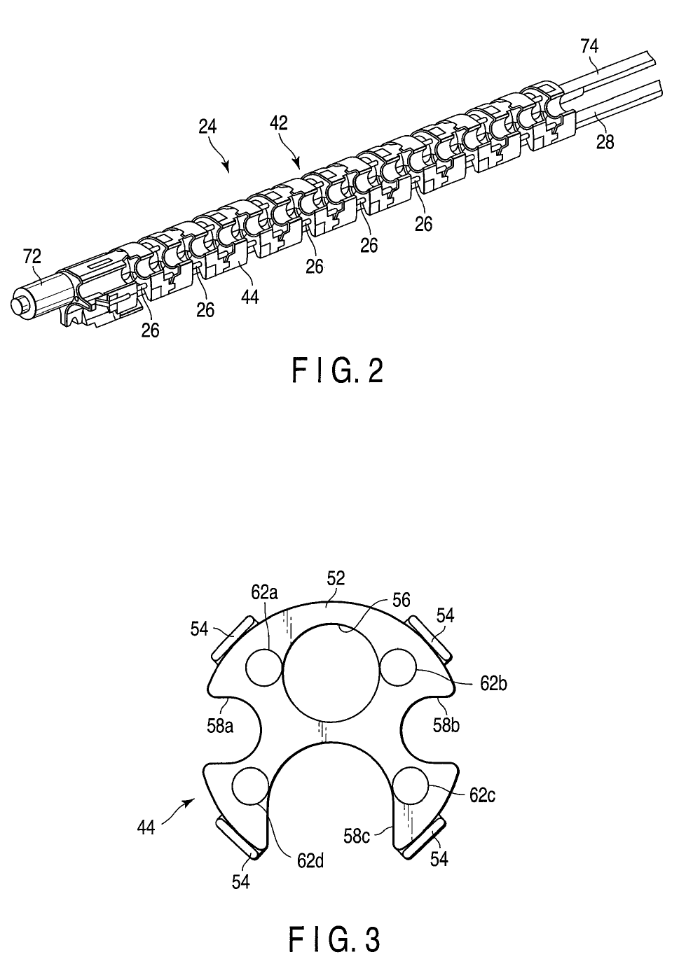

As shown in FIGS. 1A and 2, the cover-adapted endoscope 12 includes an observation optical system 22, a bending portion 24, one pair or two pairs of operation wires 26, one pair or two pairs of wire guides 28, and an operation portion 30. The operation wires 26 are inserted through the wire guides 28 respectively, and are axially moved when the bending state of the bending portion 24 is changed.

The cover-adapted endoscope 12 has the bending portion 24 on its distal end side. The operation wires 26 inserted through the wire guides 28 are disposed in the bending portion 24. Although not shown, the distal end of the operation wire 26 is fixed to the distal end of the bending portion 24. Further, the operation wires 26 and the wire guides 28 through which the operation wires 26 are inserted extend on the proximal end side of the operation portion 30. As shown in FIG. 1A, the operation portion 30 is provided on the proximal end side of the operation wires 26 and the wire guides 28. Moreover, the observation optical system 22 allows the bending portion 24 to be inserted therethrough, and is inserted through the operation portion 30.

As shown in FIG. 2, the bending portion 24 includes a bending pipe 42 capable of bending. The bending pipe 42 has a plurality of joint pieces 44 that are axially joined together. In addition, a mesh tube (not shown) is provided outside the bending pipe 42 when necessary.

As shown in FIG. 3, each of the joint pieces 44 includes a solid main body 52, joints 54 to be coupled to the adjacent joint piece 44, an annular aperture (imaging cable hole) 56 and concave grooves (air supply tube groove, water supply tube groove and suction tube groove) 58a, 58b, 58c.

The main body 52 of the joint piece 44 is in the shape of a solid thin circular plate made of a rigid material such as stainless steel or a rigid plastic material. Here, suppose that the circumference of the main body 52 of the joint piece 44 is circular and that a proper position with respect to the central axis of the main body 52 of the joint piece 44 is defined as zero degrees. In this case, with respect to the central axis of the main body 52, the joints 54 are formed at positions of 0 and 180 degrees on one side and at positions of 90 and 270 degrees on the other side. Then, pins or the like are put in holes (not shown) of the joints 54 of the adjacent joint pieces 44 that are disposed in a predetermined direction, thereby coupling the joint pieces 44 together. A plurality of joint pieces 44 are thus joined together so that the bending pipe 42 having one central axis is formed in the bending portion 24.

Furthermore, the annular aperture 56 and the concave grooves (air supply tube groove, water supply tube groove and suction tube groove) 58a, 58b, 58c are formed at positions off the central axis of the main body 52 of the joint piece 44.

In the aperture 56, a later-described imaging cable 74 of the observation optical system 22 is disposed. For example, the suction tube groove 58c is formed on the outer peripheral edge located opposite to the aperture 56 across the central axis of the main body 52. The air supply tube groove 58a and the water supply tube groove 58b are formed on the outer peripheral edge of the main body 52 between the aperture 56 and the suction tube groove 58c. The air supply tube groove 58a, the water supply tube groove 58b and the suction tube groove 58c are cuts which are concavely formed in the outer periphery of the main body 52 of the joint piece 44. Thus, the grooves 58a, 58b, 58c of the joint piece 44 are not annular, and are open on their external sides with respect to the central axis of the main body 52. In addition, an air supply tube 142, a water supply tube 144 and a suction tube 146 which are fixed to a later-described cover distal portion 112 of the endoscope-cover 14 are insertably provided in the air supply tube groove 58a, the water supply tube groove 58b and the suction tube groove 58c, respectively. That is, the air supply tube groove 58a, the water supply tube groove 58b and the suction tube groove 58c serve as guide portions for guiding the air supply tube 142, the water supply tube 144 and the suction tube 146, respectively.

First to fourth wire insertion holes 62a, 62b, 62c, 62d are formed in the main body 52 of each joint piece 44. The wire insertion holes 62a, 62b, 62c, 62d are formed at positions inside and adjacent to the joints 54. Specifically, the wire insertion holes 62a, 62b, 62c, 62d are formed at positions of about 0, about 90, about 180 and about 270 degrees with respect to the central axis of the joint piece 44 on which the joints 54 are formed. In particular, the first wire insertion hole 62a is formed between the aperture 56 of the main body 52 of each joint piece 44 and the air supply tube groove 58a. The second wire insertion hole 62b is formed between the aperture 56 and the water supply tube groove 58b. The third wire insertion hole 62c is formed between the water supply tube groove 58b and the suction tube groove 58c. The fourth wire insertion hole 62d is formed between the suction tube groove 58c and the air supply tube groove 58a.

Furthermore, for the first to fourth wire insertion holes 62a, 62b, 62c, 62d, deviations of about 5 to 10 degrees from 0, 90, 180 and 270 degrees are allowed. Such deviations of the wire insertion holes 62a, 62b, 62c, 62d are allowed from the following reason: For example, when the joint piece 44 turns about the adjacent joint piece 44 using the joints 54 located at the positions of 0 and 180 degrees, there is a small effect (force exerted when the joints 54 turn) on the joints 54 located at the positions of 90 and 270 degrees even if the positions of the first and third wire insertion holes 62a, 62c have deviated from the positions of 0 and 180 degrees.

Furthermore, the operation wires 26 and the wire guides 28 are inserted through the first to fourth wire insertion holes 62a, 62b, 62c, 62d. In addition, the distal ends of the operation wires 26 are fixed to, for example, the most distal joint piece 44 of the bending pipe 42. Moreover, the proximal ends of the operation wires 26 further extend toward the proximal side through the most proximal joint piece 44 of the bending pipe 42, and are coupled pair by pair to a later-described UD-knob 96a and RL-knob 96b of the operation portion 30.

When the bending portion 24 is formed by the operation wires 26, the wire guides 28 and the joint pieces 44, that is, when the adjacent joint pieces 44 are joined together by the joints 54 to form the bending pipe 42, the concave grooves 58a, 58b, 58c of the joint pieces 44 align in the longitudinal direction of the bending pipe 42 and form a groove structure.

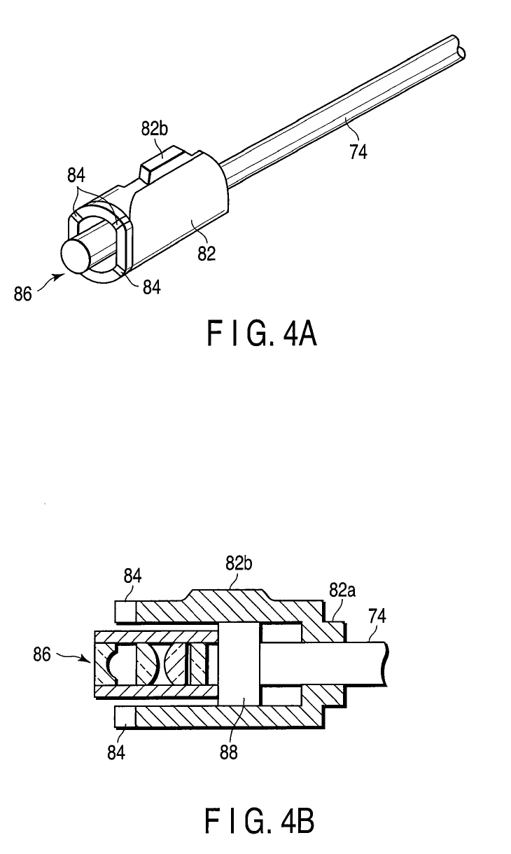

As shown in FIGS. 4A and 4B, the observation optical system 22 includes a distal imaging unit 72, and the imaging cable 74 extending from the distal imaging unit 72.

The distal imaging unit 72 includes a cylindrical main body 82 having a bottom, a small light source 84 such as an LED, an objective optical system 86 and a solid-state image sensing device 88. The solid-state image sensing device 88 includes, but not exclusively in particular, a charge-coupled device (CCD) and a complementary metal oxide semiconductor (CMOS).

The light sources 84 are provided in, for example, four places at the distal end of the main body 82 of the distal imaging unit 72. The objective optical system 86 is provided within the main body 82, and the solid-state image sensing device 88 is provided on the optical axis of the objective optical system 86. From the solid-state image sensing device 88, the imaging cable 74 extends through the bottom of the main body 82. Thus, a specimen is illuminated with light emitted from the small light sources 84, and this illumination light enters the objective optical system 86 so that an image is formed. Then, this image is picked up by the solid-state image sensing device 88 and photoelectrically transferred, and relevant information is transmitted to an unshown controller or the like through the imaging cable 74.

A circular-ring projection 82a is formed at the proximal end of the main body 82 of the distal imaging unit 72 so that this projection 82a may be fitted into and thereby fixed to the most distal joint piece 44 of the bending pipe 42. The projection 82a of the main body 82 of the distal imaging unit 72 is thus fitted into the most distal joint piece 44 of the bending pipe 42, so that the movement of the distal imaging unit 72 at the distal end of the bending pipe 42 is prevented. Moreover, a protrusion 82b is formed in the main body 82 of the distal imaging unit 72, and this protrusion 82b is fixed to the later-described cover distal portion 112 to be removable therefrom in a condition in which the direction of the distal imaging unit 72 is regulated. As a result, the distal imaging unit 72 is prevented from turning about the axis of the cover distal portion 112 during the observation of the specimen. That is, an observation image is prevented from automatically turning, for example, when the imaging cable 74 is subjected to the bending of the bending pipe 42, the operation wires 26 and the wire guides 28 during the observation of the specimen.

The imaging cable 74 extending from the distal imaging unit 72 extends from the proximal end of the bending pipe 42 through the aperture 56 (see FIG. 3) of the main body 52 of each joint piece 44. Further, the extending end of the imaging cable 74 is fixed to a connector through the operation portion 30 and a universal cord.

As shown in FIG. 1A, the operation portion 30 includes a main body 92 also serving as a grip portion to be gripped by a user of the covered-type endoscope 10, an endoscope side coupling portion 94 to be coupled to a later-described operation portion coupling portion 120 of the endoscope-cover 14, and the UD-knob 96a and the RL-knob 96b that are provided in the main body 92. Although not shown, the universal cable having the connector at its proximal end is provided to extend from the main body 92 of the operation portion 30.

The endoscope side coupling portion 94 is provided in the main body 92. A pair of operation portion coupling grooves 94a are formed in the endoscope side coupling portion 94 to be fitted by and thus coupled to operation portion coupling claws 182 of the later-described operation portion coupling portion 120 of the endoscope-cover 14.

Furthermore, an air supply tube coupling opening 98a, a water supply tube coupling opening (not shown) and a suction tube coupling opening (channel tube coupling opening) 98c are provided in the endoscope side coupling portion 94. The air supply tube coupling opening 98a is provided at the distal end of the air supply tube (not shown) which is provided through the operation portion 30 and the universal cable. The water supply tube coupling opening is provided at the distal end of the water supply tube (not shown) which is provided through the operation portion 30 and the universal cable. The suction tube coupling opening 98c is provided at the distal end of the suction tube (not shown) which is provided through the operation portion 30 and the universal cable.

The operation wires 26 are coupled to the UD-knob 96a and the RL-knob 96b within the main body 92 of the operation portion 30. Thus, when the UD-knob 96a and the RL-knob 96b are turned, the operation wires 26 inserted through the wire guides 28 move within the bending pipe 42 of the bending portion 24, and the bending portion 24 is thus bent.

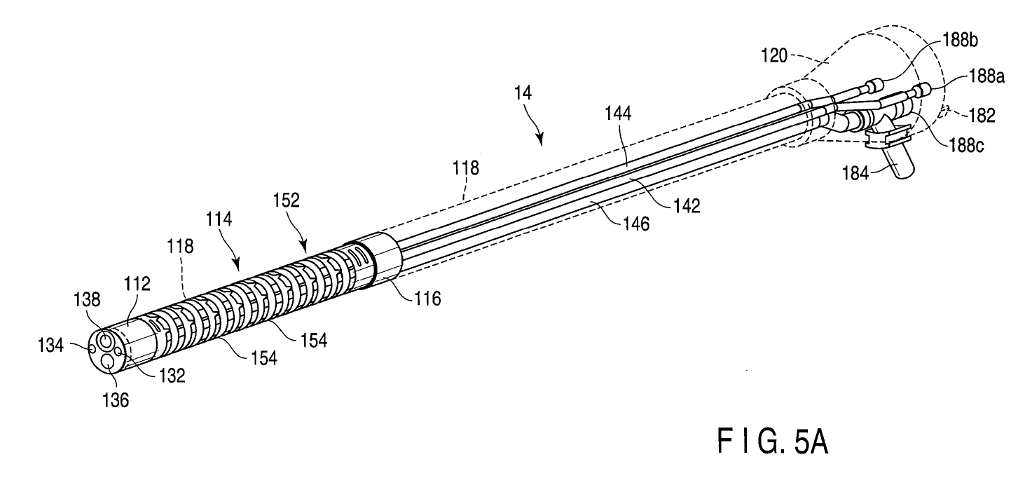

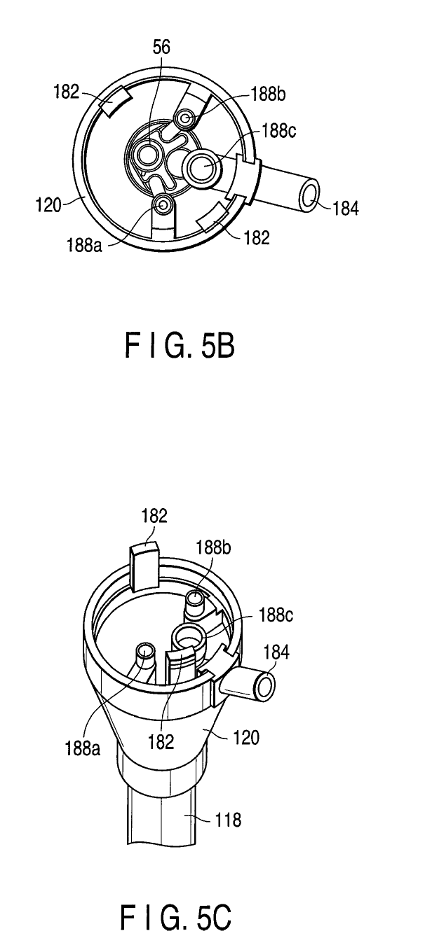

FIGS. 5A to 5C show the schematic configuration of the endoscope-cover 14 according to this embodiment.

As shown in FIG. 5A, the endoscope-cover 14 includes the cover distal portion 112, a cover bending portion 114, a cover connecting portion 116, a cover flexible pipe 118 and the operation portion coupling portion (coupling fit member) 120 from the distal side to proximal side in order.

The endoscope-cover 14 includes the rigid cover distal portion 112 at the distal end. A channel opening 132, an air supply nozzle 134, a water supply nozzle 136 and an imaging unit cover (observation port) 138 are provided in the front surface of the cover distal portion 112. The air supply tube 142, the water supply tube 144 and the suction tube 146 are coupled to the cover bending portion 114 side of the cover distal portion 112 and extend to the operation portion coupling portion 120. The air supply tube 142, the water supply tube 144 and the suction tube 146 are made of a flexible material such as PTFE.

The cover bending portion 114 which is passively bent is provided at the proximal end of the cover distal portion 112. In addition, the cover bending portion 114 easily bends in accordance with the cover flexible pipe 118.

The cover bending portion 114 includes a cover bending pipe 152 having a plurality of axially joined cover joint pieces 154, and a flexible outer tube 156 for covering the outside of the cover bending pipe 152. The outer tube 156 is made of a resin material such as a rubber material.

The cover joint piece 154 includes a ring-shaped main body 162 made of a rigid material such as stainless steel or a rigid plastic material, and a joint 164 formed integrally with the main body 162. The adjacent cover joint pieces 154 are turnably supported on each other by pins provided in the joints 164. The most distal cover joint piece 154 is fixed to the cover distal portion 112.

The cover connecting portion 116 which connects the cover bending portion 114 to the cover flexible pipe 118 is coupled to the proximal end of the outer tube 156 of the cover bending portion 114. The most proximal cover joint piece 154 of the cover bending pipe 152 is fixed to the cover connecting portion 116.

In addition, the proximal end of the outer tube 156 of the cover bending portion 114 is provided outside the cover connecting portion 116, and the distal end of the outer tube 156 is provided outside the cover distal portion 112.

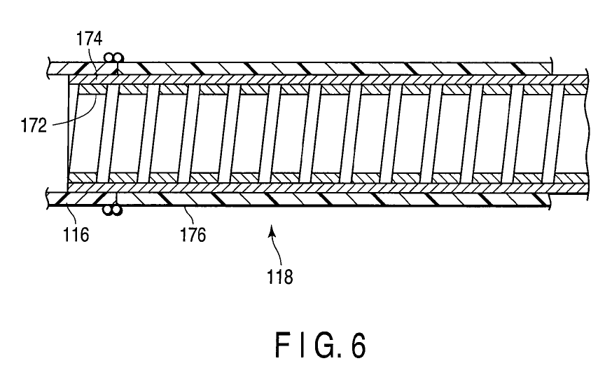

The cover flexible pipe 118 is provided at the proximal end of the cover connecting portion 116. As shown in FIG. 6, the cover flexible pipe 118 includes a flex 172, a braid 174 and an outer tube 176.

Furthermore, as shown in FIGS. 5A to 5C, the cylindrical operation portion coupling portion 120 is provided at the proximal end of the cover flexible pipe 118. The operation portion coupling portion 120 has the operation portion coupling claws 182 formed in two opposing places to be coupled to the operation portion 30 of the cover-adapted endoscope 12. Further, a surgical instrument insertion opening 184 is formed in the operation portion coupling portion 120. Moreover, a tube coupling member (pipe coupling member) 186 is provided within the operation portion coupling portion 120.

The tube coupling member 186 includes an air supply tube coupling opening 188a, a water supply tube coupling opening 188b and a suction tube coupling opening 188c. The air supply tube coupling opening 188a is attachable to and detachable from the air supply tube coupling opening 98a (see FIG. 1A) on the operation portion 30 side of the cover-adapted endoscope 12. The water supply tube coupling opening 188b is attachable to and detachable from the water supply tube coupling opening (not shown) on the operation portion 30 side of the cover-adapted endoscope 12. The suction tube coupling opening 188c is attachable to and detachable from the suction tube coupling opening 98c (see FIG. 1A) on the operation portion 30 side of the cover-adapted endoscope 12. The surgical instrument insertion opening 184 communicates with the suction tube 146. That is, the suction tube 146 is also used as a surgical instrument insertion channel.

Furthermore, FIG. 5B shows a front view of the endoscope-cover 14 viewed from the side of the operation portion 30. The cover flexible pipe 118, the cover bending portion 114 and the cover distal portion 112 through which the cover-adapted endoscope 12 is inserted are located in the center of the operation portion coupling portion 120.

Next, the function of the covered-type endoscope 10 according to this embodiment is described.

When the covered-type endoscope 10 is used, the cover-adapted endoscope 12 is inserted through and attached to the endoscope-cover 14. In this case, the distal end of the distal imaging unit 72 in the observation optical system 22 of the cover-adapted endoscope 12 shown in FIG. 2 is inserted in a predetermined direction from the operation portion coupling portion 120 of the endoscope-cover 14 shown in FIG. 5B to the cover distal portion 112 through the cover flexible pipe 118 and the cover bending portion 114.

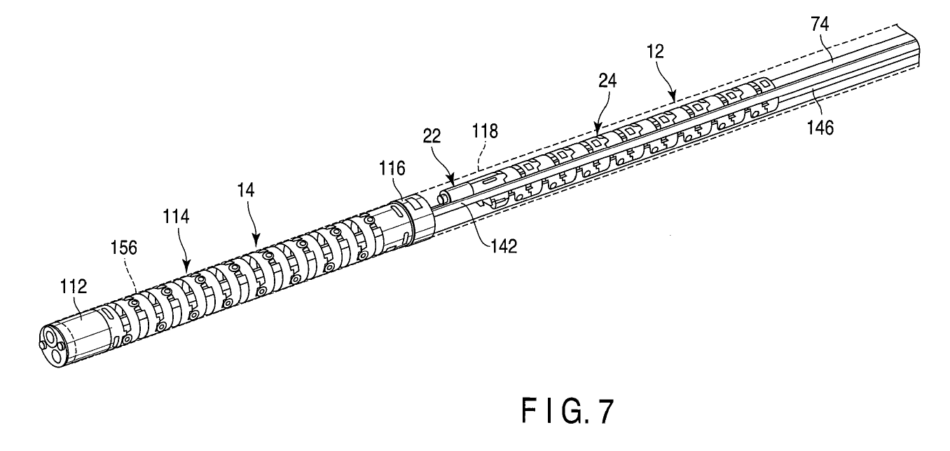

When the cover-adapted endoscope 12 is thus inserted into the endoscope-cover 14, tubes (tubular members), for example, the air supply tube 142, the water supply tube 144 and the suction tube 146 of the endoscope-cover 14 are disposed in the air supply tube groove 58a, the water supply tube groove 58b and the suction tube groove 58c of the bending portion 24 of the cover-adapted endoscope 12, as shown in FIG. 7. Thus, the air supply tube 142, the water supply tube 144 and the suction tube 146 of the endoscope-cover 14 serve as guides for disposing the cover-adapted endoscope 12 in the endoscope-cover 14.

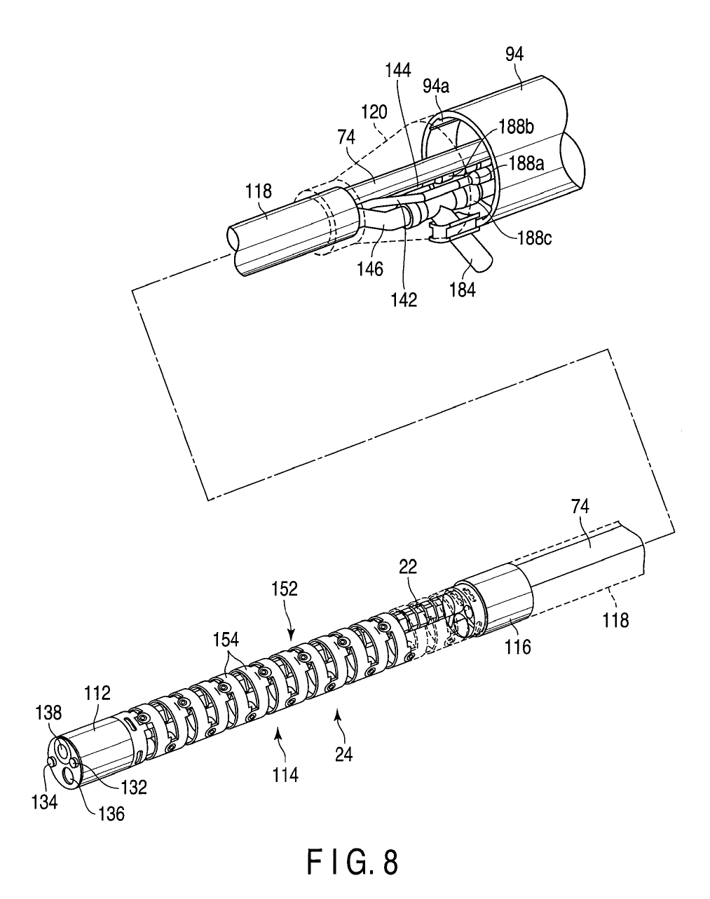

Then, as shown in FIG. 8, the endoscope side coupling portion 94 of the operation portion 30 of the cover-adapted endoscope 12 is coupled to the operation portion coupling portion 120 of the endoscope-cover 14. This coupling is completed when the operation portion coupling claws 182 of the endoscope-cover 14 are fitted in the operation portion coupling grooves 94a of the endoscope side coupling portion 94 of the operation portion 30 of the cover-adapted endoscope 12. Simultaneously with this coupling, the air supply tube coupling opening 98a (see FIG. 1A), the water supply tube coupling opening (not shown) and the suction tube coupling opening 98c (see FIG. 1A) of the cover-adapted endoscope 12 are coupled to the air supply tube coupling opening 188a, the water supply tube coupling opening 188b and the suction tube coupling opening 188c of the endoscope-cover 14. That is, the air supply tube 142, the water supply tube 144 and the suction tube 146 of the endoscope-cover 14 communicate with the air supply tube, the water supply tube and the suction tube of the endoscope-cover 14 of the cover-adapted endoscope 12 that are not shown.

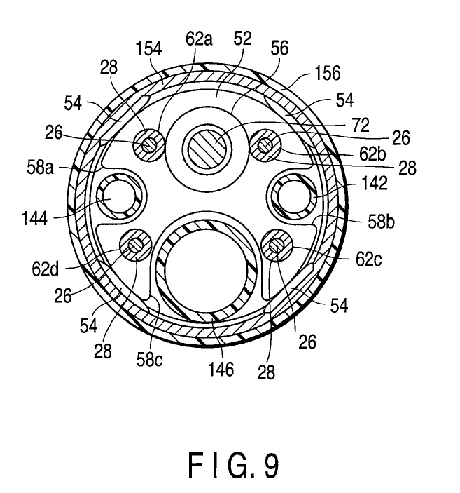

Furthermore, as shown in FIG. 9, the distal imaging unit 72 is contained in the cover distal portion 112, and the bending pipe 42 of the bending portion 24 of the cover-adapted endoscope 12 is disposed within the cover bending pipe 152 of the cover bending portion 114. Then, the air supply tube 142, the water supply tube 144 and the suction tube 146 are accommodated in a space (tube placement chamber) which is formed by the bending pipe 42 of the cover-adapted endoscope 12 and the cover bending pipe 152 of the endoscope-cover 14.

The insertion work for the covered-type endoscope 10 is thus completed (see FIG. 1B), and the covered-type endoscope 10 is ready for use.

When the cover bending portion 114 of the endoscope-cover 14 is bent, the UD-knob 96a and the RL-knob 96b of the operation portion 30 of the cover-adapted endoscope 12 are operated. As a result of this operation, the operation wires 26 are axially pulled and released and are thus moved. Then, the joint pieces 44 of the bending pipe 42 of the bending portion 24 are subjected to force from the operation wires 26. As a result, the bending portion 24 of the cover-adapted endoscope 12 bends. Further, as shown in FIG. 9, the central axis (longitudinal axis) of the bending pipe 42 of the bending portion 24 of the cover-adapted endoscope 12 is substantially coincident with the central axis (longitudinal axis) of the bending pipe 152 of the cover bending portion 114 of the endoscope-cover 14. Therefore, the bending portion 24 of the cover-adapted endoscope 12 bends, and the cover bending portion 114 of the endoscope-cover 14 bends accordingly.

Here, the central axis of the bending portion 24 of the cover-adapted endoscope 12 is substantially coincident with the central axis of the cover bending portion 114 of the endoscope-cover 14. Thus, the cover bending portion 114 of the endoscope-cover 14 bends to an extent that is substantially symmetrical with respect to the central axis.

Furthermore, the joint pieces 44 of the bending pipe 42 of the cover-adapted endoscope 12 and the cover joint pieces 154 of the cover bending pipe 152 of the endoscope-cover 14 are made of a metal material such as stainless steel, titanium steel or an alloy of these metals, or made of a polyether ether ketone (PEEK) resin, a polyacetal (POM) resin, a polytetrafluoroethylene (PTFE) resin, another fluorine-based resin or a resin including a fluorine-based resin, or made of a rigid material such as reinforced plastic. Therefore, even when the bending pipe 42 and the cover bending pipe 152 are bent, the space (tube placement chamber) accommodating the flexible tubes 142, 144, 146 is substantially maintained along the bending shape. Thus, deformation such as the crushing of the tubes 142, 144, 146 is prevented. This prevents functions such as air supply, water supply and suction from being affected by the clogging of a conduit resulting from the bending of the tubes 142, 144, 146.

After use of the endoscope 10 (after surgery), the operation portion coupling grooves 94a of the endoscope side coupling portion 94 of the cover-adapted endoscope 12 are disengaged with the operation portion coupling claws 182 of the operation portion coupling portion 120 of the endoscope-cover 14. Then, the cover-adapted endoscope 12 is pulled from the endoscope-cover 14. Thus, the air supply tube coupling opening 98a, the water supply tube coupling opening (not shown) and the suction tube coupling opening 98c of the endoscope side coupling portion 94 of the cover-adapted endoscope 12 are also disengaged with the air supply tube coupling opening 188a, the water supply tube coupling opening 188b and the suction tube coupling opening 188c of the operation portion coupling portion 120 of the endoscope-cover 14. At the same time, as shown in FIG. 7, the air supply tube 142, the water supply tube 144 and the suction tube 146 of the endoscope-cover 14 serve as guides for removing the cover-adapted endoscope 12 from the endoscope-cover 14.

Moreover, as shown in FIG. 1A, the cover-adapted endoscope 12 and the endoscope-cover 14 are completely separated from each other, and then the endoscope-cover 14 is disposed of as a used endoscope-cover. On the other hand, the cover-adapted endoscope 12 is simply cleaned, disinfected and sterilized for reuse.

As described above, the following can be said according to this embodiment.

When the cover-adapted endoscope 12 and the endoscope-cover 14 are coupled together, the air supply tube coupling opening 188a, the water supply tube coupling opening 188b and the suction tube coupling opening 188c of the endoscope-cover 14 are respectively coupled to the air supply tube coupling opening 98a, the water supply tube coupling opening (not shown) and the suction tube coupling opening 98c on the operation portion 30 side of the cover-adapted endoscope 12. Thus, the cover-adapted endoscope 12 and the endoscope-cover 14 can be easily positioned relative to each other when the cover-adapted endoscope 12 and the endoscope-cover 14 are coupled together. Further, the endoscope side coupling portion 94 on the operation portion 30 side of the cover-adapted endoscope 12 is provided with the operation portion coupling grooves 94a, and the operation portion coupling portion 120 on the endoscope-cover 14 side is provided with the operation portion coupling claws 182, so that the positioning for coupling is enabled by fitting the operation portion coupling claws 182 into the operation portion coupling grooves 94a at the time of coupling. This fitting makes it possible to prevent the coupling portion of the cover-adapted endoscope 12 and the endoscope-cover 14 from being axially and circumferentially unstable (e.g., wobbling) during the use of the covered-type endoscope 10.

Furthermore, the joint pieces 44 of the bending pipe 42 of the cover-adapted endoscope 12 and the cover joint pieces 154 of the cover bending pipe 152 of the endoscope-cover 14 are made of a rigid material. Therefore, even when the bending pipe 42 and the cover bending pipe 152 are bent, the space accommodating the flexible tubes 142, 144, 146 can be substantially maintained along the bending shape. Thus, deformation such as the crushing of the tubes 142, 144, 146 can be prevented. This prevents the air supply, water supply and suction functions from being affected when the bending pipe 42 and the cover bending pipe 152 are bent.

Consequently, it is possible to provide the covered-type endoscope 10 capable of having operability as well as the air supply, water supply and suction functions during bending.

Although the operation wires 26 are inserted through the wire guides 28 within the bending portion 24 in the embodiment described above, the operation wires 26 do not always have to be inserted through the wire guides 28 within the bending portion 24. When the operation wires 26 are not inserted through the wire guides 28 within the bending portion 24, the distal ends of the wire guides 28 are fixed to, for example, the proximal end of the bending portion 24.

### Second Embodiment

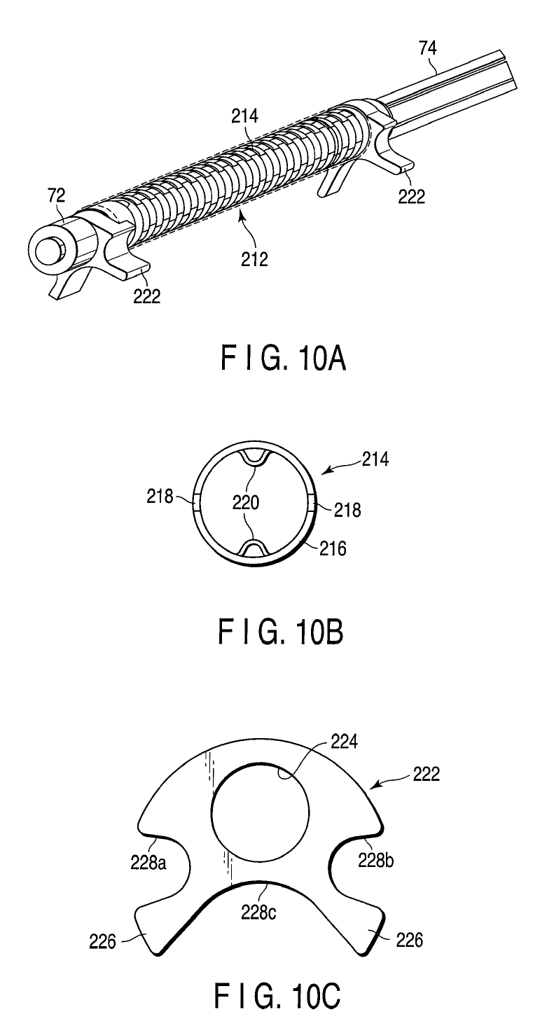

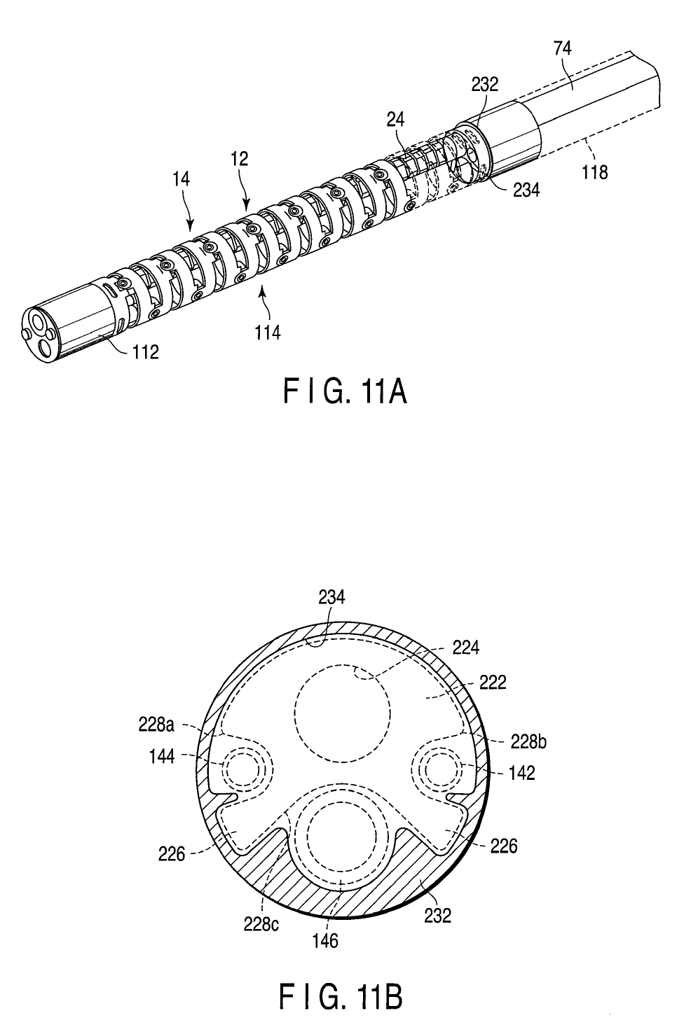

Next, a second embodiment is described with reference to FIGS. 10A to 11B. This embodiment is a modification of the first embodiment, and the same parts as the parts described in the first embodiment are provided with the same reference numbers and are not described in detail.

FIG. 10A shows the structure of a bending portion 24 of a cover-adapted endoscope 12 according to the second embodiment corresponding to FIG. 2 described in the first embodiment.

In contrast with the shape of the joint piece 44 described in the first embodiment, a joint piece 214 of a bending pipe 212 of the bending portion 24 of the cover-adapted endoscope 12 has a ring-shaped main body 216 as shown in FIG. 10B, similarly to the cover joint piece 154 of the cover bending pipe 152 of the endoscope-cover 14. Further, two pairs of joints 218 (only one pair is shown) and a pair of wire receivers 220 are formed in the main body 216. Pins are provided in the joints 218 of the adjacent joint pieces 214, so that the adjacent joint pieces 214 are pivotally supported on each other. Moreover, the wire receivers 220 are formed inside the main body 216 of each of the joint pieces 214. The wire receivers 220 receive operation wires 26 or wire guides 28 through which the operation wires 26 are inserted.

Furthermore, spacers (guide portions) 222 are fixed to the distal and proximal ends of the bending pipe 212 of the bending portion 24 of the cover-adapted endoscope 12. That is, the most distal and most proximal joint pieces 214 of the bending pipe 212 of the bending portion 24 of the cover-adapted endoscope 12 are fixed to the spacers 222, respectively. As shown in FIG. 10C, each of the spacers 222 includes an annular aperture (imaging cable hole) 224, a plurality of feet 226 and a plurality of concave grooves (air supply tube groove, water supply tube groove and suction tube groove) 228a, 228b, 228c.

An imaging cable 74 of the observation optical system 22 is provided in the annular aperture 224. Between the feet 226, the air supply tube groove 228a and the water supply tube groove 228b, for example, are formed face to face with each other. Further, between the air supply tube groove 228a and the water supply tube groove 228b, the suction tube groove 228c is formed across the feet 226.

On the other hand, a cover connecting portion 232 is a member for connecting a cover bending portion 114 to a cover flexible pipe 118 of the endoscope-cover 14, in place of the cover connecting portion 116 in the first embodiment shown in FIG. 8. In contrast with the cover connecting portion 116 that is simply in a substantially cylindrical shape, the cover connecting portion 232 has an inner peripheral surface 234 which is shaped so that the feet 226 of the spacers 222 are positioned and so that the shapes of the air supply tube 142, the water supply tube 144 and the suction tube 146 are maintained, as shown in FIG. 11B. Although not shown, the inner peripheral surface of a cover distal portion 112 of the endoscope-cover 14 is also shaped so that the spacers 222 are positioned and so that the shapes of the air supply tube 142, the water supply tube 144 and the suction tube 146 are maintained, similarly to the inner peripheral surface 234 of the cover connecting portion 232.

Furthermore, the inside diameter of the cover joint piece 154 of the cover bending portion 114 of the endoscope-cover 14 is substantially coincident with the maximum inside diameter from the center of the cover connecting portion 232. That is, the distal ends (distal ends with respect to the imaging cable 74 of the observation optical system 22 in FIG. 10A) of the feet 226 come into contact with at least the inner surfaces of the cover joint pieces 154 of the cover bending portion 114 of the endoscope-cover 14 when the cover-adapted endoscope 12 is inserted through the endoscope-cover 14. That is, the air supply tube groove 228a, the water supply tube groove 228b, the suction tube groove 228c are formed in the spacer 222 to guide the air supply tube 142, the water supply tube 144 and the suction tube 146.

Moreover, the joint piece 44 of the bending portion 24 shown in FIG. 2 in the first embodiment has a diameter close to the inside diameter of the cover bending portion 114 of the endoscope-cover 14 (see FIG. 9). On the other hand, in this embodiment, the joint piece 44 has a diameter about ⅔ to ½ of the inside diameter of the cover bending portion 114 of the endoscope-cover 14. Therefore, the ratio (A/B) of the “inside diameter of the joint piece 44 of the bending portion 24 of the cover-adapted endoscope 12” (defined as A) to the “inside diameter of the cover joint piece 154 of the cover bending portion 114 of the endoscope-cover 14” (defined as B) is smaller in this embodiment than in the first embodiment, and closer to “1” in the first embodiment.

The following can be said according to this embodiment.

When the cover-adapted endoscope 12 is inserted into the endoscope-cover 14 to couple an endoscope side coupling portion 94 of the cover-adapted endoscope 12 to an operation portion coupling portion 120 of the endoscope-cover 14, the spacers 222 provided at the distal and proximal ends of the bending portion 24 of the cover-adapted endoscope 12 can be substantially engaged with the inner peripheries of the cover distal portion 112 and the cover connecting portion 232 of the endoscope-cover 14. Thus, the circumferential movement of the spacers 222 with respect to the endoscope-cover 14 can be regulated. As a result, the spacers 222 of the cover-adapted endoscope 12 can be positioned with respect to the circumferential direction of the endoscope-cover 14.

When a UD-knob 96a and an RL-knob 96b of an operation portion 30 of the cover-adapted endoscope 12 are operated to bend the bending portion 24 of the cover-adapted endoscope 12 and also bend the cover bending portion 114 of the endoscope-cover 14, the spacers 222 come into contact with the inside diameter of the endoscope-cover 14, and the inside diameter of the cover bending portion 114 can be ensured. Thus, deformation such as the crushing of the tubes 142, 144, 146 is prevented.

Furthermore, by the grooves 228a, 228b, 228c formed between the feet 226 of the spacer 222, spaces where the air supply tube 142, the water supply tube 144 and the suction tube 146 are provided can be formed between the outer peripheries of the air supply tube 142, the water supply tube 144 and the suction tube 146 and the inner peripheral surfaces of the grooves 228a, 228b, 228c of the spacer 222 and the cover connecting portion 232. This makes it possible to prevent deformation such as the crushing of the air supply tube 142, the water supply tube 144 and the suction tube 146 between the pair of spacers 222 at the distal and proximal ends of the bending portion 24 of the cover-adapted endoscope 12.

Therefore, even when the cover bending portion 114 of the endoscope-cover 14 is disposed outside the bending portion 24 of the cover-adapted endoscope 12 and then the bending portion 24 and the cover bending portion 114 are bent, deformation such as the crushing of the air supply tube 142, the water supply tube 144 and the suction tube 146 can be prevented.

Although the spacer 222 at the distal end of the bending portion 24 of the cover-adapted endoscope 12 is separate from a distal imaging unit 72 of the observation optical system 22 in this embodiment described above, the spacer 222 may be integrated with the distal imaging unit 72. In this case, no positioning member (e.g., the protrusion indicated by reference number 82b in FIGS. 4A and 4B) is necessary for the distal imaging unit 72.

### Third Embodiment

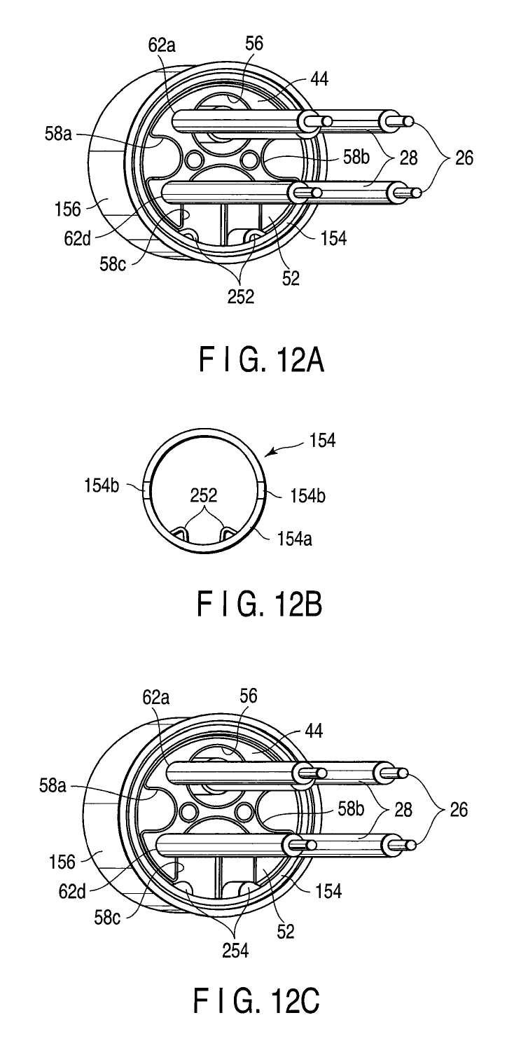

Next, a third embodiment is described with reference to FIGS. 12A to 12C. This embodiment is a modification of the first embodiment, and the same parts as the parts described in the first embodiment are provided with the same reference numbers and are not described in detail.

This embodiment is intended to ensure that twisting force (torque) about the axis of a bending portion 24 of a cover-adapted endoscope 12 is transmitted to the side of an endoscope-cover 14 and to ensure that twisting force about the axis of a cover bending portion 114 of the endoscope-cover 14 is transmitted to the side of the cover-adapted endoscope 12.

FIG. 12A shows the internal structure of a bending pipe 42 of the bending portion 24 when the endoscope-cover 14 is attached to the cover-adapted endoscope 12. A cover joint piece 154 of the endoscope-cover 14 includes a main body 154a and a joint 154b. A pair of torque transmission protrusions (twisting force transmission means) 252 are formed inside the main body 154a of the cover joint piece 154 by, for example, cutting and bending of the main body 154a. These torque transmission protrusions 252 are formed at positions to engage with, for example, the suction tube groove (twisting force transmission means) 58c of the joint piece 44 shown in FIG. 3. In addition, the torque transmission protrusions 252 have only to be designed so that the twisting force of an operation portion 30 of the cover-adapted endoscope 12 is transmitted to the endoscope-cover 14 and so that the twisting force of the cover bending portion 114 of the endoscope-cover 14 is transmitted to the cover-adapted endoscope 12. Therefore, shaking between the joint piece 44 and the torque transmission protrusions 252 is allowed.

The cover-adapted endoscope 12 is longitudinally positioned with respect to the endoscope-cover 14 by disposing a distal imaging unit 72 of an observation optical system 22 of the cover-adapted endoscope 12 in the cover distal portion 112 of the endoscope-cover 14. Moreover, the cover-adapted endoscope 12 is circumferentially positioned with respect to the endoscope-cover 14 by the engagement (contact) of the suction tube groove 58c of the most proximal joint piece 44 of the cover-adapted endoscope 12 with the torque transmission protrusion 252 provided in the most proximal cover joint piece 154 of a cover bending pipe 152 of the cover bending portion 114 of the endoscope-cover 14. Then, when the bending portion 24 of the cover-adapted endoscope 12 is bent and when the cover bending portion 114 of the endoscope-cover 14 is bent in accordance with the bending portion 24 of the cover-adapted endoscope 12, the joint piece 44 of the bending portion 24 of the cover-adapted endoscope 12 and the cover joint piece 154 of the cover bending portion 114 of the endoscope-cover 14 move together in the longitudinal direction of the torque transmission protrusion 252. Therefore, the suction tube groove 58c of the joint piece 44 of the bending portion 24 of the cover-adapted endoscope 12 is positioned incorrectly relative to the torque transmission protrusions 252 of the cover joint piece 154 of the cover bending portion 114 of the endoscope-cover 14. However, the torque transmission protrusion 252 has a longitudinally extended shape to maintain the engagement of the torque transmission protrusion 252 with the suction tube groove 58c of the joint piece 44 of the bending portion 24 of the cover-adapted endoscope 12 when the bending portion 24 of the cover-adapted endoscope 12 and the cover bending portion 114 of the endoscope-cover 14 are bent. Thus, the cover-adapted endoscope 12 is prevented from being circumferentially out of position with respect to the endoscope-cover 14 when the bending portion 24 of the cover-adapted endoscope 12 and the cover bending portion 114 of the endoscope-cover 14 are bent.

Furthermore, the operation portion 30 of the cover-adapted endoscope 12 is coupled to an operation portion coupling portion 120 of the endoscope-cover 14, thereby ensuring that torque is transmitted to each other in accordance with the engagement between the bending portion 24 of the cover-adapted endoscope 12 and the cover bending portion 114 of the endoscope-cover 14.

The following can be said according to this embodiment.

In the present embodiment, the torque transmission protrusions 252 are provided on the inner peripheral surface of the cover joint piece 154 of the bending portion 114 of the endoscope-cover 14. Thus, for example, when the operation portion 30 of the cover-adapted endoscope 12 is twisted to turn the operation portion coupling portion 120, the bending portion 114 of the endoscope-cover 14 does not come out of position with respect to the bending portion 24 of the cover-adapted endoscope 12, thereby ensuring that torque can be transmitted. Moreover, when a cover flexible pipe 118 of the endoscope-cover 14 is twisted, torque can be transmitted to the operation portion 30 of the cover-adapted endoscope 12.

Consequently, when the operation portion 30 of a covered-type endoscope 10 according to this embodiment is turned about the axis of the endoscope-cover 14, the toque is also transmitted to the bending portion 114 of the endoscope-cover 14. Moreover, when twisting force is applied to the cover bending portion 114 of the endoscope-cover 14, the twisting force can be transmitted to the bending portion 24 and the operation portion 30 of the cover-adapted endoscope 12. Thus, the covered-type endoscope 10 can obtain good operability equal to that of an endoscope which is reused after being cleaned, disinfected and sterilized.

In addition, the torque transmission protrusion 252 may have a protrusion (twisting force transmission means) 254 produced by, for example, a forging process as shown in FIG. 12C instead of having the cut and bent shape shown in FIGS. 12A and 12B. In this case, similar effects can also be obtained.

While several embodiments have been specifically described so far with reference to the drawings, this invention is not limited to the embodiments described above and includes all embodiments without departing from the spirit thereof.

What is claimed is:

1. A covered-type endoscope comprising:
an endoscope-cover including a tube and a cover-bending portion with a plurality of longitudinally joined cover-joint pieces; and
a cover-adapted endoscope to be inserted into the endoscope-cover, the cover-adapted endoscope including a bending portion with a plurality of longitudinally joined joint pieces, the bending portion including a guide portion which guides the tube in the longitudinal direction of the bending portion and cooperates with the cover-bending portion of the endoscope-cover to provide a tube placement chamber between the guide portion and the cover-bending portion of the endoscope-cover so that a conduit for the tube is maintained;
wherein the tube is flexible to bend in accordance with the bending of the cover-bending portion and the bending portion.

2. The covered-type endoscope according to claim 1, wherein a wire which bends the bending portion of the cover-adapted endoscope is passed through the joint pieces of the cover-adapted endoscope.

3. The covered-type endoscope according to claim 1, wherein the guide portion of the bending portion of the cover-adapted endoscope includes a concave portion in the joint piece to dispose the tube therein.

4. The covered-type endoscope according to claim 1, wherein the guide portion of the bending portion of the cover-adapted endoscope includes a spacer in the bending portion of the cover-adapted endoscope to dispose the tube therein.

5. The covered-type endoscope according to claim 1, wherein:
the cover-adapted endoscope includes an operation portion to bend the bending portion, and a conduit coupling opening provided in the operation portion, and
a tube coupling member is detachably provided in a part of the tube of the endoscope-cover to be coupled to the conduit coupling opening of the operation portion.

6. The covered-type endoscope according to claim 1, wherein the endoscope-cover includes a coupling fit member in a part to be coupled to the operation portion of the cover-adapted endoscope.

7. The covered-type endoscope according to claim 1, wherein the cover-joint piece of the cover bending portion of the endoscope-cover includes twisting force transmission means for transmitting twisting force to the cover-adapted endoscope.

8. The covered-type endoscope according to claim 7, wherein
the twisting force transmission means includes a protrusion directed toward the cover-adapted endoscope, and
the joint piece of the bending portion of the cover-adapted endoscope includes a concave portion which receives the protrusion.

9. The covered-type endoscope according to claim 2, wherein the guide portion of the bending portion of the cover-adapted endoscope includes a concave portion in the joint piece to dispose the tube therein.

10. The covered-type endoscope according to claim 2, wherein the guide portion of the bending portion of the cover-adapted endoscope includes a spacer in the bending portion of the cover-adapted endoscope to dispose the tube therein.

11. The covered-type endoscope according to claim 2, wherein:
the cover-adapted endoscope includes an operation portion to bend the bending portion, and a conduit coupling opening provided in the operation portion, and
a tube coupling member is detachably provided in a part of the tube of the endoscope-cover to be coupled to the conduit coupling opening of the operation portion.

12. The covered-type endoscope according to claim 2, wherein the endoscope-cover includes a coupling fit member in a part to be coupled to the operation portion of the cover-adapted endoscope.

13. The covered-type endoscope according to claim 2, wherein the cover-joint piece of the cover bending portion of the endoscope-cover includes twisting force transmission means for transmitting twisting force to the cover-adapted endoscope.

14. The covered-type endoscope according to claim 13, wherein
the twisting force transmission means includes a protrusion directed toward the cover-adapted endoscope, and
the joint piece of the bending portion of the cover-adapted endoscope includes a concave portion which receives the protrusion.

15. A cover-adapted endoscope to be let into or out of an endoscope-cover which includes a flexible tube configured to follow a bending operation, the cover-adapted endoscope comprising:
a bending portion to be inserted into the endoscope-cover, the bending portion including a guide portion which guides the tube in the longitudinal direction of the bending portion and cooperates with the endoscope-cover to form a tube placement chamber between the guide portion and the endoscope-cover so that a conduit for the tube is maintained; and
an operation portion provided at the proximal end of the endoscope-cover.

16. An endoscope-cover which allows a cover-adapted endoscope including a bending portion to be let into or out of the endoscope-cover, the endoscope-cover comprising:
a cover-bending portion which is disposed outside the bending portion of the cover-adapted endoscope when the cover-adapted endoscope is inserted in the endoscope-cover;
a cover distal end portion provided at the distal end of the cover-bending portion; and
a tube which extends from the cover distal end portion toward a side of the cover bending portion, the tube being flexible to bend in accordance with the bending of the cover-bending portion and the bending portion of the cover-adapted endoscope, and cooperates with the cover-adapted endoscope to form a tube placement chamber between the cover bending portion and the cover-adapted endoscope so that a conduit for the tube is maintained.
